(19) United States
(12) Patent Application Publication
(10) Pub. No.: US 2008/0319515 A1
(43) Pub. Date: Dec. 25, 2008

(54) PROCESS FOR REDUCING NEUROMUSCULAR FATIGUE CAUSED BY EXERCISE

(76) Inventors: Alberto Priori, Virgilio (Mantova) (IT); Filippo Maria Cogiamanian, Parabiago (Milano) (IT); Sara Renata Francesca Marceglia, Milano (IT)

Correspondence Address: PEARNE & GORDON LLP, 1801 EAST 9TH STREET, SUITE 1200, CLEVELAND, OH, 44114-3108, US

(21) Appl. No.: 11/766,401

(22) Filed: Jun. 21, 2007

(51) Int. Cl. A61N 1/20

(52) U.S. Cl. 607/75

(57) ABSTRACT

A process for reducing neuromuscular fatigue caused by exercise comprises applying an anodal trans-cranial direct current stimulation over the right motor cortex of a person.

The present invention relates to a process for reducing neuromuscular fatigue caused by exercise, in particular for reducing fatigue in a person about to participate in exercise or in person having completed exercise.

Neuromuscular fatigue is the exercise dependent decrease in muscle ability to generate force. Therefore, in the context of the present invention, the term “fatigue” has to be intended as a condition, induced by the performance of exercises, which is a transitory physiological condition caused by a natural circumstance.

Simple training is generally known as retarding the perception of fatigue.

There also exist natural or synthetic drugs that reduce the fatigue caused by exercise, nevertheless many of such drugs involve addiction and cause adverse side effects.

The aim of the present invention is therefore to provide an alternative process for reducing neuromuscular fatigue caused by exercise.

This aim is achieved by the present invention, which involves manipulating brain excitability by anodal trans-cranial direct current stimulation, as disclosed in one or more of the appended claims.

An anodal trans-cranial direct current stimulation is a process of electric stimulation of a cortical region of a person's scalp with an anodal direct current of low intensity, namely of low milliAmpere.

In particular, the process comprises applying an anodal trans-cranial direct current stimulation over the motor cortical areas of a person.

It has been found that the anodal trans-cranial direct current stimulation, described in detail below, improves muscle endurance, helping to start increased muscle endurance and decreasing muscle fatigue in normal, namely sports medicine, conditions.

The anodal trans-cranial direct current stimulation comprises applying an anodal electrode on the scalp of a person over the motor cortical areas.

Advantageously the anodal electrode is to be centred on the motor cortical areas of the scalp possibly to be active on both the right and the left motor cortical areas themselves.

It has been verified that good results may be also achieved placing the electrode specifically on the motor cortex.

In particular, in the test phase the anodal electrode was applied on the right motor cortex and in detail 4 centimetres laterally to the vertex.

A cathodal electrode is applied on a portion of the body of the same person; generally such an electrode is placed on a portion other than the scalp.

Preferably, the cathodal electrode is applied above the shoulder and, during the experimental tests, precisely on the right shoulder.

A direct current is then flowed from the cathodal electrode to the anodal electrode for a period between 5 and 60 minutes (in relation to the intensity used). Anyway a current flowing for a period between 5 to 30 minutes is generally sufficient.

The direct current intensity is chosen between 0.5 to 5 milliAmpere (in relation to the duration used), in order to polarise the brain, but a preferred upper limit is 3 milliAmpere.

The intensity and the standing of the direct current depend on various parameters, such as the physical features of the person, the position of the muscle on which the process has to be implemented and other.

In the preferred embodiment of the present invention, the intensity of the direct current is between 1 to 2 milliAmpere, preferably of 1.5 milliampere, for a period between 8 to 12 minutes, preferably of 10 minutes.

The direct current can be delivered by any suitable electrical device, for example by an electrical stimulator through a constant current unit and an isolation unit connected to said electrodes.

It is important that the direct current is an anodal current, since cathodal current does not achieve appreciable effects on the reduction of neuromuscular fatigue.

The electrodes may have any desirable shape even if a round shape gives better results with respect to squared or irregular shapes.

Of course a conductive material, such as a conductive gel or a saline composition, has to be used in combination with the electrodes.

The used electrodes have pieces of saline-soaked synthetic sponge in order to avoid possible harmful effects of high current density.

The preferred dimension for each electrode is a thickness of 0.3 centimetres and an area of 35 square centimetres even if other dimensions may be suitable as well.

In this regard, the direct current flowing from the cathodal electrode to the anodal electrode has a charge density that is between 0.01 to 0.5 Coulomb/square centimetre.

The range upper limit will be generally maintained under 0.2 Coulomb/square centimetre and more preferably the charge density is between 0.01 to 0.05 Coulomb/square centimetre; in the implemented method a charge density of 0.026 Coulomb/square centimetre was used.

Hereinafter the implementation of the process according to the present invention as performed on a group of 24 people is reported, by evaluating the fatigue reduction of left elbow flexors caused by exercise. Said group consisted of 24 healthy right handed volunteers, in particular 14 women and 10 men having a mean age 24.3 years. None of the subjects engaged in competitive sport activities specifically involving elbow flexor muscles. The experimental procedures were in accordance with the declaration of Helsinki.

Subjects sat in a chair with the left elbow on a padded support, the elbow joint at a right angle, and the wrist connected to a load cell. The motor task required the subject to exert an upward directed force with the wrist activating the elbow flexor muscles.

To determine the maximal voluntary contraction force, subjects were instructed to increase the force from zero to a maximum level and to hold it for 3 seconds maximal voluntary contraction. Subjects performed nine maximal voluntary contraction trials subdivided in three sessions and rested for 60 sec between each trial. The mean of the best three performances was taken as the maximal voluntary contraction force and used as the reference to calculate the target force for the fatigue contraction.

Muscle endurance was assessed as the time subjects could sustain an isometric contraction with the elbow flexor muscles at 35% of the maximal voluntary contraction force as determined previously. During the fatigue task, each subject received force feedback on a computer monitor showing a 35% maximal voluntary contraction target. The subject was encouraged to keep the force at this level.

The contraction was terminated when the subject deviated from the target force for more than 3 seconds despite strong verbal encouragement.

Trans-cranial magnetic stimulation was delivered by a stimulator through a flat coil centred over the vertex with currents flowing clockwise (viewed from above). Motor evoked potentials were recorded by standard non-polarizable Ag—AgCl surface electrodes placed over the belly of the biceps brachii muscle and on the skin overlying the biceps' tendon of the left arm. The motor threshold was defined as the lowest intensity able to produce motor evoked potentials of ≧50 μV in 5 out of 10 consecutive trials of stimulation during a slight contraction of biceps. Stimulation intensity was 120% of the baseline motor evoked potential threshold. A total of 16 motor evoked potentials were recorded in response to 16 stimuli delivered at 0.15 Hz. The peak-to-peak amplitude was measured before and immediately after trans-cranial direct current stimulation.

The trans-cranial direct current stimulation had an intensity of 1.5 milliAmpere and a duration of 10 minutes. It was delivered by an electrical stimulator through a constant current unit and an isolation unit connected to a pair of electrodes. One electrode was placed on the scalp over the right motor cortex, in particular 4 cm laterally to the vertex and the other electrode was placed above the right shoulder. Stimulating electrodes were pieces of saline-soaked synthetic sponge. To guarantee safety, the density charge was of 0.026 C/cm². These criteria are far below the threshold for tissue damage. The wide electrode surface avoided the possible harmful effects of high current density. Apart from occasional, transient and short lasting tingling and burning sensations below the electrodes, direct current stimulation strength remained below the conscious cutaneous sensory threshold throughout the experimental session. Direct current stimulation polarity (cathodal or anodal) refers to the electrode over the right motor area.

The effect of anodal and cathodal trans-cranial direct current stimulation on maximal voluntary contraction and endurance time was evaluated. Three levels, namely anodal trans-cranial direct current stimulation, cathodal trans-cranial direct current stimulation and independent measures without stimulation were performed.

No difference was noted between normalized endurance time values at 1 hour after cathodal trans-cranial direct current stimulation and measures without stimulation. Anodal trans-cranial direct current stimulation induced a significant decrease in endurance time at 1 hour when compared to measures without stimulation and cathodal trans-cranial direct current stimulation.

In particular, at 1 hour after the baseline fatigue task, the expected shortening of the endurance time, owing to residual tiredness, is reduced by about 15% after anodal trans-cranial direct current stimulation compared with cathodal trans-cranial direct current stimulation and measures without stimulation.

The example above disclosed shows that anodal trans-cranial direct current stimulation applied over the right motor cortical areas prolongs the endurance time for contralateral elbow flexors in a submaximal isometric task.

Analogous effects are achieved on all the other muscles of the people subjected to the anodal trans-cranial direct current stimulation, in particular when the direct current has been applied over the right motor cortical areas.

Moreover, anodal trans-cranial direct current stimulation improves muscle performance and decreasing muscle fatigue both in normal and pathological conditions, for example in stroke and primary or secondary chronic fatigue syndrome rehabilitation.

1. Process for reducing neuromuscular fatigue caused by exercise comprising applying an anodal trans-cranial direct current stimulation over the motor cortical areas of a person.

2. Process according to claim 1 comprising applying an anodal electrode on a scalp of a person over the motor cortex.

3. Process according to claim 1 comprising applying a cathodal electrode on a portion of the body of the same person and then flowing a direct current between said electrodes.

4. Process according to claim 3 comprising making a direct current flow from the cathodal electrode to the anodal electrode; said direct current being between 0.5 to 5 milliAmpere for a period between 5 to 60 minutes.

5. Process according to claim 3 comprising applying said cathodal electrode on the right shoulder.

6. Process according to claim 2 comprising applying said anodal electrode on both the right and left cortical areas of the person.

7. Process according to claim 3 comprising flowing said direct current having a charge density comprised between 0.01 to 0.5 Coulomb/square centimetre.

8. Process according to claim 3 comprising flowing said direct current having a charge density comprised between 0.01 to 0.05 Coulomb/square centimetre.

9. Process according to claim 4 wherein the direct current is between 1 to 2 milliAmpere for a period between 8 to 12 minutes.

10. Process according to claim 9 wherein the direct current is of 1.5 milliAmpere for a period of 10 minutes.

11. Process according to claim 8 comprising flowing said direct current having a charge density of 0.026 Coulomb/square centimetre.

12. Process according to claim 2 comprising providing said electrode having a round shape.

13. Process according to claim 2 comprising providing said electrode having pieces of saline-soaked synthetic sponge.

14. Process according to claim 3 comprising applying a cathodal electrode on a portion of the body of the same person other than the scalp.

15. Process for treating chronic neuromuscular fatigue comprising applying an anodal electrode on the scalp of a patient over the motor cortical areas, applying a cathodal electrode on a portion of the body of the same patient, making a direct current flow from the cathodal electrode to the anodal electrode; said direct current having a charge density comprised between 0.01 to 0.5 Coulomb/square centimetre.

16. Process according to claim 15 wherein the cathodal electrode is applied on a portion of the body of the same patient other than the scalp.

17. Process according to claim 15 wherein said direct current has a charge density comprised between 0.01 to 0.05 Coulomb/square centimetre and the direct current is between 0.5 to 5 milliAmpere for a period between 5 to 30 minutes.
